(19) United States
(12) Patent Application Publication
(10) Pub. No.: US 2007/0187475 A1
(43) Pub. Date: Aug. 16, 2007

(54) Medication verification systems and methods

(76) Inventor: Ronald R. MacLeod, Mississauga (CA)

Correspondence Address: BERESKIN AND PARR, 40 KING STREET WEST, BOX 401, TORONTO, ON, M5H 3Y2, CA

(21) Appl. No.: 11/341,565

(22) Filed: Jan. 30, 2006

(51) Int. Cl. A61B 5/00; G06K 7/10; G06F 17/00; G06Q 30/00

(52) U.S. Cl. 235/375; 235/462.01; 600/300; 235/385

(57) ABSTRACT

Human errors during the administration of medications to patients results in a number of accidents that can be attributed to not having a systematic method for verifying the contents of secondary containers used for storing and administering medications. Some embodiments of the invention provide an electronically-aided systems and methods for validating the contents of a secondary container after a medication has been transferred from a primary container to the secondary container and before the medication is administered to a patient. In accordance with some aspects of the invention, labels for secondary containers are provided. These labels are referred to as User Applied Medication Labels (UAML) and these labels are different from conventions medication vial labels because they are applied by health care professionals onto secondary containers. In some embodiments the UAML include machine readable information corresponding to the machine-readable information on the labels on primary containers supplied by the pharmaceutical companies.

### FIELD OF THE INVENTION

The invention relates to medical informatics, and in particular to systems, methods and apparatus for safely administering medications.

### BACKGROUND OF THE INVENTION

Pharmaceutical companies often package medications in liquid form in containers such as vials or ampoules. For the sake of brevity, the containers that pharmaceutical companies use to package medications in are referred to as primary containers hereinafter. A label on a primary container often includes a machine-readable barcode and/or RFID (Radio Frequency IDentification) tag to aid in the identification of the contents of the primary container in addition to human-readable text and symbols. The barcodes and/or RFID tags store information such as Drug Identification Numbers (DIN), UPC codes and National Drug Codes (that are used specifically in the United States).

Within a health-care facility a medication in liquid form is often transferred from a primary container to a secondary container. Secondary containers include, for example and without limitation, syringes, cups, solution bowls and basins. Secondary containers are used to temporarily store and/or administer medications.

For example, in an operating theatre, medications are brought into a sterile field around a patient using syringes and/or another type of secondary container. Each syringe may contain a different medication for use during the operation. Most medications are clear and colorless, so it is almost impossible to simply identify the contents of a secondary container (e.g. a syringe) without some type of visual aid.

Accordingly, medical professionals employ a number of ad hoc methods for identifying medications in secondary containers. For example, specific medications are sometimes paired with a specific size and/or type of secondary container. However, such practices are not standardized and different medical professionals often pair medications with secondary containers differently from their colleagues. In another example, a secondary container is provided with a label and/or color-code indicia. However, user applied labels are often quite small and the text on the labels can be smudged or covered by blood (or, other fluids) that may cause the user applied labels to be misread. As a result of the ad hoc methods employed, numerous patients are incorrectly administered medications causing a number of side effects ranging from the relatively harmless to loss of vital organ function and sometimes even death.

Human error is the primary source of the errors made. However, human error in a health-care environment is difficult to address, since medical staff act according to strict operating procedures that are hard to adjust without introducing added liability. Subsequently, medical staff are often averse to procedural changes because such changes are thought to inherently include increased liability.

Additionally, within an inpatient health-care facility, such as a nursing home or a hospital, nurses routinely administer medication(s) to patients as prescribed by a doctor and/or on an as needed basis. In many jurisdictions nurses are required to record the details relating to the distribution of the medications in order to comply with regional health care regulations. The details may include a listing of medications provided, time, reason, outcome (i.e. observations) and dosage of medication(s) provided to each patient.

The workflow described above is widely susceptible to human error, as there are few points at which the activities of individuals can be checked to ensure that individuals (e.g. nurses) working within an inpatient health-care facility are complying with regional health care regulations.

For example, while nurses are supposed to record the time and dosage of medications at the same time the medications are administered to a patient, some nurses first distribute medications to a number of patients and then update and initial the patient-specific charts, thereby separating the tasks of distribution and recordation. This practice can lead to accidentally providing the wrong medication to one or more patients, providing the correct medication at the wrong times, failing to provide the medication to a patient and/or misjudging the effects of particular medications on respective patients. In more extreme cases, a nurse may actually distribute medication before or after the respective prescribed time, but nevertheless update a patient-specific chart as though the medication was provided at the appropriate time. Additionally, nurses may forget to document the time, reason and outcome for providing medication that is to be administered to a particular patient only as needed, since scheduled times for providing such medication are not listed on a patient-specific chart. Unfortunately, there is no practical way for anyone else to detect that an individual in charge of distributing medications at particular times is not following health-care regulations set out by the inpatient health-care facility and/or a governing body (e.g. a state and/or federal agency).

### SUMMARY OF THE INVENTION

According to an aspect of an embodiment of the invention there is provided a system for verifying the contents of a secondary container, the secondary container suitable for storing and administering medications in a liquid form, the system comprising: a User Applied Medication Label (UAML), for a secondary container, including machine-readable information corresponding to machine-readable included on a primary container for a medication in liquid form; a machine-scanner for scanning machine-readable information and providing a scanned output; and a workstation computer connectable to the machine-scanner for receiving the scanned output from the machine-scanner.

In some embodiments, the UAML includes a barcode containing the machine-readable information. Additionally and/or alternatively, the UAML includes a RFID tag containing the machine-readable information.

In some embodiments the machine-readable information includes at least one of a Drug Identification Number, a UPC code and a National Drug Code.

In some embodiments the workstation computer includes a computer usable program code including program instructions for: receiving scanned machine-readable information from a primary container and a secondary container; comparing the scanned machine-readable information from the primary container and the secondary container; providing affirmative feedback if the primary and secondary containers have the same scanned machine-readable information; and providing dissenting feedback if the primary and secondary containers do not have the same scanned machine-readable information.

Additionally and/or alternatively, in some embodiments the computer usable program code further includes program instructions for creating a record of the comparison. Additionally and/or alternatively, in some embodiments the computer usable program code including program instructions for updating a database of records, wherein each record contains the results and a particular comparison.

Additionally and/or alternatively, in some embodiments the UAML are sterilized. Additionally and/or attentively, in some embodiments the sterilized UAML are packaged in a sterilized package suitable for use in an operating theatre.

According to an aspect of an embodiment of the invention there is provided a method for verifying the contents of a secondary container, the secondary container suitable for storing and administering medications in a liquid form, the method comprising: scanning machine-readable information from a primary container containing a medication in liquid form; scanning machine-readable information for a secondary container for use in temporarily storing and then administering medication; comparing the scanned machine-readable information from the primary container and the secondary container; providing affirmative feedback if the primary and secondary containers have the same scanned machine-readable information; and providing dissenting feedback if the primary and secondary containers do not have the same scanned machine-readable information.

Additionally and/or alternatively, in some embodiments the method further comprising: preparing the medication in the primary container according to a set of safe standard operating practices; and transferring the medication from the primary to secondary container.

In some more specific embodiments, preparing the medication includes verifying and recording at least one of the type of medication, dosage, patient, time and correct route.

Other aspects and features of the present invention will become apparent, to those ordinarily skilled in the art, upon review of the following description of the specific embodiments of the invention.

### BRIEF DESCRIPTION OF THE DRAWINGS

For a better understanding of the present invention, and to show more clearly how it may be carried into effect, reference will now be made, by way of example, to the accompanying drawings, which illustrate aspects of embodiments of the present invention and in which:

FIG. 1 is a schematic drawing of a system for verifying the contents of secondary containers in a clinical environment; and

FIG. 2 is a flow chart depicting the general steps of an electronically-aided method for verifying the contents of secondary containers in a clinical environment.

### DETAILED DESCRIPTION OF THE INVENTION

Changing the operating procedures within a health-care facility may inadvertently introduce new sources of liability. Subsequently, health-care professionals are naturally very cautious and risk averse when contemplating the adoption of new operating procedures and electronic systems that involve significant changes to their accepted procedures. However, human errors during the distribution of medications to patients results in a number of accidents that can be attributed to not having a systematic method for verifying the contents of secondary containers used for storing and administering medications.

Some embodiments of the invention provide electronically-aided systems and methods for validating the contents of a secondary container after a medication has been transferred from a primary container to the secondary container and before the medication is administered to a patient. In accordance with some aspects of the invention, labels for secondary containers are provided. These labels are referred to as User Applied Medication Labels (UAML) and these labels are different from conventional medication primary container labels because the UAML's are applied by health care professionals onto secondary containers. In some embodiments the UAML include machine readable information corresponding to the machine-readable information on the labels on primary containers supplied by the pharmaceutical companies.

In accordance with some embodiments, the machine-readable information may be stored in barcode form and/or within an RFID tag. The barcodes and/or RFID tags store information such as Drug Identification Numbers (DIN), UPC codes and National Drug Codes (that are used specifically in the United States). In some embodiments the same information is also typically printed on the labels for visual identification.

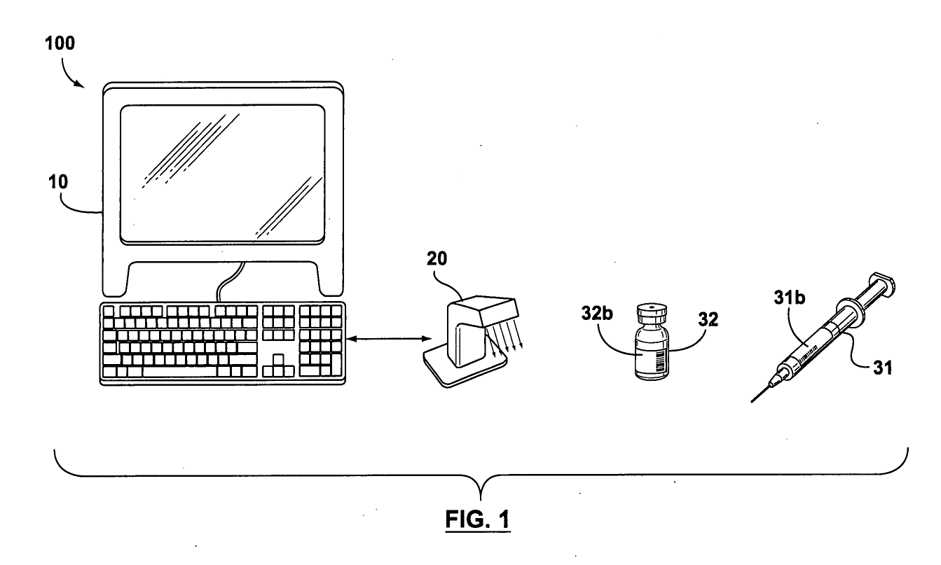

In accordance with some aspects of the invention a system is provided for verifying the contents of a secondary container. With reference to FIG. 1, shown is a schematic diagram of an example system 100 in accordance with aspects of the invention. The system 100 includes workstation computer 10 (e.g. a personal computer, network computer, etc.), and a machine-scanner 20 coupled to the workstation computer 10. The workstation computer 10 includes a processor and a memory that is accessible by the processor. Those skilled in the art will appreciate that the workstation computer 10 also includes an additional suitable combination of hardware, software and firmware, and that the functional elements illustrated in FIG. 1 have only been provided to describe aspects of a very specific embodiment of the invention.

In some embodiments the machine-scanner 20 is suitable to scan information from barcodes. Additionally and/or alternatively, the machine-scanner 20 is suitable to scan information from RFID tags.

Also shown in FIG. 1, for the sake of example only, is a vial of medication 32 (the primary container) and a syringe 31 (the secondary container). The primary container 32 has a label 32b with machine-readable information, as described above. Similarly, the secondary container 31 also has a label 31b with machine-readable information.

In operation, the system 100 is used to scan the labels 32b and 31b to verify whether or not the information on each is the same. The machine-scanner 20 is used to scan each label 32b and 31b individually and provide the information to the workstation computer 10. The workstation computer 10, having received the scanned information on both labels compares the scanned information and provides feedback to a user. If the labels are the same, the feedback is affirmative. On the other hand if the information is not the same the feedback is dissenting and the user is instructed to not administer the medication in the secondary container to a patient.

Moreover, by scanning the labels 31b and 32b and storing the result of the comparison a record is created that can be stored in the memory of the workstation computer 10. In a hospital or another health-care facility, records of all medications provided to respective patients can be created in this way. Each such record can be reviewed at a later time to verify that the individual(s) responsible for preparing and/or administering the medications was (were) being diligent while preparing and/or administering the medications. That is, in accordance with aspects of the invention, the record created by scanning labels on primary and secondary containers can be used as a metric for quality control and culpability.

A number of records created by the system can be stored in a database. To that end, aspects of the invention may be embodied in a number of forms. For example, various aspects of the invention can be embodied in a suitable combination of hardware, software and firmware. In particular, some embodiments include, without limitation, entirely hardware, entirely software, entirely firmware or some suitable combination of hardware, software and firmware. In a preferred embodiment, the invention is implemented in software, which includes but is not limited to firmware, resident software, microcode, etc.

Additionally and/or alternatively, aspects of the invention can be embodied in the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

A computer-readable medium can be an electronic, magnetic, optical, electromagnetic, infrared, or semiconductor system (or apparatus or device) or a propagation medium. Examples of a computer-readable medium include a semiconductor and/or solid-state memory, magnetic tape, a removable computer diskette, a random access memory (RAM), a read-only memory (ROM), a rigid magnetic disk and an optical disk. Current examples of optical disks include, without limitation, compact disk—read only memory (CD-ROM), compact disk−read/write (CD-R/W) and DVD.

In accordance with aspects of the invention, a data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output (i.e. I/O devices)—including but not limited to keyboards, displays, pointing devices, etc.—can be coupled to the system either directly or through intervening I/O controllers.

Network adapters may also be coupled to the system to enable communication between multiple data processing systems, remote printers, or storage devices through intervening private or public networks. Modems, cable modems and Ethernet cards are just a few of the currently available types of network adapters.

In some embodiments the “User Applied Medication Labels” are sterilized using gamma radiation. The sterilized labels are then packaged in a sterile environment to ensure that the labels remain sterile so that they may be safely introduced into a sterile field in an operating theatre. Additionally, in some embodiments secondary containers include syringes, solution bowls, basins, medicine cups or similar vessels used to temporarily store and/or administer a medication.

In accordance with some aspects of the invention, the “User Applied Medication Labels” allow for the replacement or rotation of health care staff during the administration of medication. For example, during a long surgical procedure, a nurse can be relieved for breaks or lunch. Systems and methods provided by aspects of the invention provide at least some assurance to the relieving nurse that the medications on the sterile field are correct because the relieving nurse can validate the contents of secondary containers by scanning the “User Applied Medication Labels”.

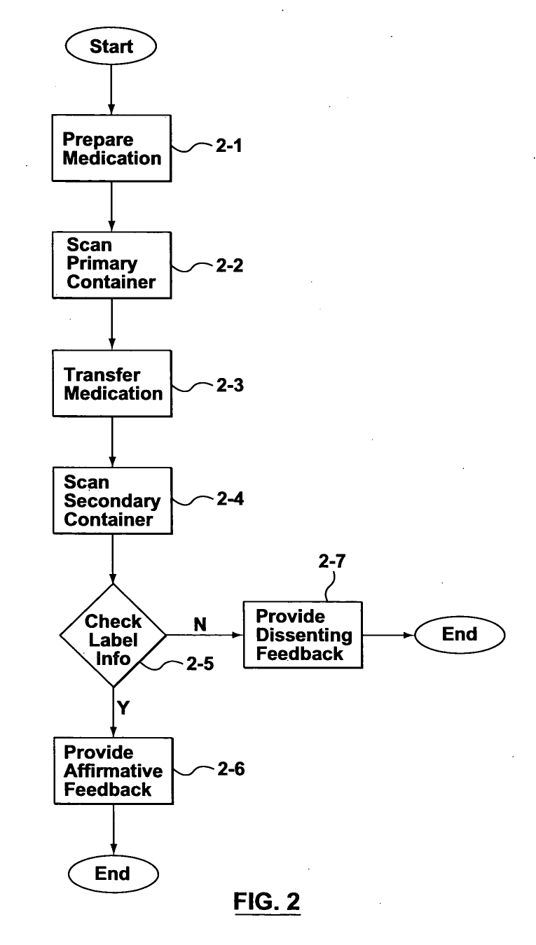

Referring to FIG. 2, shown is a flow chart depicting the general steps of an electronically-aided method for verifying the contents of secondary containers in a clinical environment. Starting at step 2-1, a medical professional (i.e. a user) prepares medications as per hospital standard and regional health-care regulations. In some cases, this may include verifying: that the medications to be administered are correct (with reference to instructions from a doctor); dosage; patient; time; and, correct route as per normal standards of practice.

At step 2-2, the label on a primary container (e.g. a vial) for a medication is scanned and the scanned information is stored. Then at step 2-3, the medication is transferred from the primary container to a secondary container labeled for the medication. At step 2-4, the secondary container is scanned and the corresponding scanned information is stored.

At step 2-5, the scanned information from the primary and secondary containers is compared to ensure that the information is the same. If the scanned information from the primary and secondary containers is the same, the user is provided with affirmative feedback at step 2-6. On the other hand, if the scanned information from the primary and secondary containers is not the same, the user is provided with dissenting feedback at step 2-7.

While the above description provides example embodiments, it will be appreciated that the present invention is susceptible to modification and change without departing from the fair meaning and scope of the accompanying claims. Accordingly, what has been described is merely illustrative of the application of aspects of embodiments of the invention. Numerous modifications and variations of the present invention are possible in light of the above teachings. It is therefore to be understood that within the scope of the appended claims, the invention may be practiced otherwise than as specifically described herein.

I claim:

1. A system for verifying the contents of a secondary container, the secondary container suitable for storing and administering medications in a liquid form, the system comprising:
a User Applied Medication Label (UAML), for a secondary container, including machine-readable information corresponding to machine-readable included on a primary container for a medication in liquid form;
a machine-scanner for scanning machine-readable information and providing a scanned output; and
a workstation computer connectable to the machine-scanner for receiving the scanned output from the machine-scanner.

2. A system according to claim 1, wherein the UAML includes a barcode containing the machine-readable information.

3. A system according to claim 1, wherein the UAML includes a RFID tag containing the machine-readable information.

4. A system according to claim 1, wherein the machine-readable information includes at least one of a Drug Identification Number, a UPC code and a National Drug Code.

5. A system according to claim 1, wherein the workstation computer includes a computer usable program code including program instructions for:
receiving scanned machine-readable information from a primary container and a secondary container;
comparing the scanned machine-readable information from the primary container and the secondary container;
providing affirmative feedback if the primary and secondary containers have the same scanned machine-readable information; and
providing dissenting feedback if the primary and secondary containers do not have the same scanned machine-readable information.

6. A system according to claim 5, wherein the computer usable program code further includes program instructions for creating a record of the comparison.

7. A system according to claim 6, wherein the computer usable program code including program instructions for updating a database of records, wherein each record contains the results and a particular comparison.

8. A system according to claim 1, wherein the UAML are sterilized.

9. A system according to claim 8, wherein the sterilized UAML are packaged in a sterilized package suitable for use in an operating theatre.

10. A method for verifying the contents of a secondary container, the secondary container suitable for storing and administering medications in a liquid form, the method comprising:
scanning machine-readable information from a primary container containing a medication in liquid form;
scanning machine-readable information for a secondary container for use in temporarily storing and then administering medication;
comparing the scanned machine-readable information from the primary container and the secondary container;
providing affirmative feedback if the primary and secondary containers have the same scanned machine-readable information; and
providing dissenting feedback if the primary and secondary containers do not have the same scanned machine-readable information.

11. A method according to claim 10, further comprising:
preparing the medication in the primary container according to a set of safe standard operating practices; and
transferring the medication from the primary to secondary container.

12. A method according to claim 11, wherein preparing the medication includes verifying and recording at least one of the type of medication, dosage, patient, time and correct route.
